(19) United States
(12) Patent Application Publication
(10) Pub. No.: US 2007/0166713 A1
(43) Pub. Date: Jul. 19, 2007

(54) System and method for fully automated robotic-assisted image analysis for in vitro and in vivo genotoxicity testing

(75) Inventor: Wilfried Frieauff, Inzlingen (DE)

Correspondence Address: DARBY & DARBY P.C., P. O. BOX 5257, NEW YORK, NY, 10150-5257, US

(73) Assignee: NOVARTIS AG, Basel (CH)

(21) Appl. No.: 10/554,520

(22) PCT Filed: Mar. 6, 2004
(86) PCT No.: PCT/IB04/00623

§ 371 Date: Mar. 22, 2006

Related U.S. Application Data
(60) Provisional application No. 60/465,564, filed on Apr. 25, 2003.

(51) Int. Cl. C12Q 1/68; G06K 9/00; C12M 1/34

(52) U.S. Cl. 435/6; 435/287.2; 382/128

(57) ABSTRACT

A system and method is provided for performing genotoxicity screening. The system and method utilize: (1) one or more computers; (2) a frame grabber connected to the one or more computers; (3) a camera connected to the frame grabber; (4) a microscope connected to the one or more computers; (5) a slide feeder connected to the one or more computers; and (6) a program operating on the one or more computers. The program facilitates the screening a second batch of biological material using a second genotoxicity testing method after screening a first batch of biological material using a first genotoxicity testing method. The screening operates substantially free of any manual manipulation of the camera, the microscope or the slide feeder.

### REFERENCE TO COMPUTER PROGRAM LISTING APPENDIX

A Computer Program Listing Appendix to this document has been submitted to the U.S. Patent and Trademark Office in accordance with 37 C.F.R. §§ 1.52 and 1.96 on the filing date of this document and is hereby incorporated herein by reference in its entirety. The Computer Program Listing Appendix is contained on one (1) CD-ROM, two copies of which have been filed with the U.S. Patent and Trademark Office and each of which are labeled with the name of the inventor of the present invention, the title of the invention, the attorney's docket number and the creation date of the CD-ROMs.

### FIELD OF THE INVENTION

The present invention is directed to genotoxicity testing and, more particularly, to a method and system for utilizing, in conjunction, an automated robotic slide feeder or equivalent device, an electronically driven microscope, a microprocessor-based computer and additional components and software to facilitate high-throughput in vitro and in vivo genotoxicity testing.

### BACKGROUND OF THE INVENTION

Toxicological testing is used in various technologies, industries and disciplines for assessing the effect of drugs and other chemical compounds on the nature and properties of biological matter. Genotoxicity testing is particularly useful for analyzing the effect of certain chemicals on the DNA structure of the cells of humans, animals and other life forms, including the analysis of the potential for induction of hereditary diseases and mutations. Genotoxicity testing generally includes screening of either in vivo or in vitro biological matter.

Well known in vitro test systems include, but are not limited to:

(1) the comet assay, which is used for detecting primary DNA damage, DNA to DNA crosslinks, and DNA-protein interactions. A specific version of the comet assay is the Alkaline Comet Assay which is described in a publication titled “A simple technique for quantiation of low levels of DNA damage in individual cells,” Singh et al., Experimental Cellular Research, vol. 175, pp. 184-191 (1988). The Alkaline Comet Assay is also described in a publication titled “Modification of the Comet Assay for the detection of DNA strand breaks in extremely small tissue samples,” Tebbs et al., Mutagenesis, vol. 14, pp. 437 438 (1999);

(2) the micronucleus test in cell lines (V79 cells, Mouse Lymphome cells, TK6 cells) or human lymphocytes, which are all known to be useful in the early screening of new compounds in industrial toxicology; and

(3) the chromosome aberration test, which is required by certain regulatory authorities, such as the Organization for Economic Co-Operation and Development and the United States Food and Drug Administration, for approval of new drugs. For this in vitro test, the assessment of chromosomal aberrations is done on the basis of metaphases which must be detected for analysis.

In vivo genotoxicity test systems include, but are not limited to:

(1) the in vivo micronucleus test in bone marrow for clastogenic or aneugenic potential of a test compound administered to rodents. This test is described in a publication titled “A Rapid in vivo test for chromosomal damage,” Heddle, J A., Mutual Res., vol. 18, pp. 187-90 (1973);

(2) the in vivo comet assay, which under certain circumstances may be accepted as a regulatory assay in addition to the micronucleus test in vivo, to verify in vitro test results. The in vivo comet assay is described in a publication titled “Recommendations for conducting the in vivo alkaline Comet assay”, Hartmann et al., Mutagenesis vol. 18, no. 1, pp. 45-51 (2003).

Other in vivo and in vitro testing methods are also well known in the art.

Limited automated methods for facilitating genotoxicity screening (“screening” being understood to refer to the analysis of biological material samples previously treated with the test compound) of both in vivo and in vitro materials have also been attempted. As an example, an automated in vivo micronucleus assay analysis of mouse bone marrow used in the pharmaceutical industry to test the genotoxicity potential of new compounds is described in a publication co-authored by the inventor of the present invention which is titled “Technical aspects of automatic micronucleus analysis in rodent bone,” Cell Biology and Toxicology, vol. 10, pp. 283-289 (1994). Automated forms of analysis for in vitro micronucleus tests are also known. The inventor of the present invention authored an article titled “Automatic analysis of the in vitro micronucleus test on V79 cells” in Mutation Research, vol. 413, pp. 57-68 (1998), describing an automated in vitro micronucleus test for V79 cells.

The techniques for automated genotoxicity screening for both in vivo and in vitro biological material that were noted above utilize image analysis software and techniques that are individually designed for the specific type of test and the specific type of material that is being screened. Automation of genotoxicity testing that utilizes image analysis simplifies the process of compound screening, eliminates the tedium of manual scoring and significantly increases the overall number of genotoxicity screenings which can be performed in any given period of time. Generally, an automated electronically driven microscope with image capturing capabilities and a micro-processor based computer running microscope control and image analysis software, each specifically designed, calibrated and programmed for the particular screening being performed, is used to operate and facilitate the image analysis-based automated screening process.

To provide still further increases in the throughput of genotoxicity sample screening, prior art devices are known to have incorporated robotic arm assemblies and equivalent devices to facilitate sample slide feeding, thus freeing the user from the tedium of manually loading slides for image analysis and further increasing screening throughput rates.

Known prior art systems do not, however, allow for both in vivo and in vitro genotoxicity screening using a single platform to perform automatically all manners of in vitro and in vivo genotoxicity testing such as the micronucleus test, the comet assay and metaphase detection for chromosome analysis, nor do any known prior art system provide for utilization of a robotic slide feeder or equivalent device for all manner of in vitro and in vivo testing without the tedium of extensive user intervention.

### SUMMARY

An embodiment of a genotoxicity screening system of the present invention includes: (1) one or more computers; (2) a frame grabber connected to the one or more computers; (3) a camera connected to the frame grabber; (4) a microscope connected to the one or more computers; (5) a slide feeder connected to the one or more computers; and (6) a program operating on the one or more computers. The program facilitates the screening a second batch of biological material using a second genotoxicity testing method after screening a first batch of biological material using a first genotoxicity testing method. The genotoxicity methods are performed substantially free of any manual manipulation of the camera, the microscope or the slide feeder.

In another embodiment of the present invention, software is provided that controls the operation of a genotoxicity analysis system. The software provides automatic configuration of configurable components of the genotoxicity analysis system and allows the genotoxicity analysis system to perform a plurality of genotoxicity tests on respective pluralities of biological samples by way of the automatic configuration.

In another embodiment of the present invention, genotoxicity testing of biological materials is performed using a genotoxicity analysis system. The genotoxicity system includes hardware components that are operated with software controls. The genotoxicity analysis system is capable of performing a multiplicity of genotoxicity tests. Use of the genotoxicity analysis system performs as follows: (1) preparing a first batch of samples of biological materials for processing using a first genotoxicity test; (2) utilizing the genotoxicity analysis system to perform a first genotoxicity test on the samples of the first batch of biological materials; (3) preparing a second batch of samples of biological materials for processing using a second genotoxicity test; and (4) utilizing the genotoxicity analysis system to perform a second genotoxicity test on the samples of the second batch of biological materials. The software controls manipulate the configuration of the hardware components during the time period between performance of the first and second genotoxicity tests to allow the first and second genotoxicity tests to be performed using the same hardware components.

Yet another embodiment of the present invention includes a method for performing various types of genotoxicity tests on respective batches of biological samples using a genotoxicity analysis system. The method including the steps of: (1) receiving a command from a user of the genotoxicity analysis system, the command specifying the type of genotoxicity test to be performed; (2) performing an automatic configuration of the component of the genotoxicity analysis system to thereby allow the genotoxicity analysis system to perform the genotoxicity test specified in step 1; (3) performing the specified genotoxicity test on a batch of biological samples; (4) recording results of the genotoxicity test; (5) repeating steps 1 through 4.

In yet another embodiment of a method for performing genotoxicity screening in accordance with the present invention, the following steps are performed: (1) preparing a batch of slides for genotoxicity screening; (2) selecting a genotoxicity test; (3) automatically retrieving the first of a plurality of slides containing biological samples from a slide retaining device; (4) automatically delivering the slide to an electronically driven microscope; (5) automatically focusing on the material contained on the slide; (6) automatically recording a visual representation of the focused image; (7) automatically delivering the focused image to a microprocessor-based computer; (8) automatically performing image analysis on the recorded image using image analysis software appropriate for the genotoxicity test selected in step 2; (9) automatically recording the data resulting from the analysis of the image; (10) automatically returning the slide retrieved in step 3 to the slide retaining device; (11) automatically retrieving the next slide for analysis; (12) automatically repeating steps 3 through 11 for successive slides in the batch until all of the slides in the batch have been analyzed; and (13) repeating steps 1 through 12 until all desired slides have been processed.

### BRIEF DESCRIPTION OF THE DRAWINGS

The foregoing and other features of the present invention will be more readily apparent from the following detailed description and drawings of illustrative embodiments of the invention in which:

FIG. 1 illustrates, in block diagram form, an embodiment of the automated genotoxicity analysis system;

FIG. 2 illustrates, in logical block diagram form, an embodiment of application software to control the operation of a genotoxicity analysis system and files which store the results of the genotoxicity analysis;

FIG. 3 illustrates a flow chart describing the operation of an embodiment of a genotoxicity analysis system;

FIG. 4 illustrates an embodiment of a user interface screen for entering information relating to a slide that is to be analyzed using a genotoxicity analysis system;

FIG. 5 illustrates an embodiment of a user interface screen for entering data identifying particular slides to be processed using a genotoxicity analysis system;

FIG. 6 illustrates an embodiment of a user interface screen for adjusting the parameters for a particular slide to be analyzed using a genotoxity analysis system;

FIG. 7 illustrates an embodiment of a user interface screen that allows a user to adjust the threshold settings for the particular slide that is to be analyzed using a genotoxicity analysis system;

FIG. 8 illustrates an embodiment of a user interface form for adjusting microscope parameters for the a particular slide using a genotoxicity analysis system;

FIG. 8a illustrates an embodiment of a user interface form for use in selecting a genotoxicity test that to be processed using a genotoxicity analysis system;

FIG. 9 illustrates an embodiment of a user interface screen for a user to select scanning options for a genotoxicity analysis system;

FIG. 10 illustrates an embodiment of a user interface screen for selecting results of a genotoxicity test for review using a genotoxicity analysis system;

FIG. 11 illustrates an embodiment of a user interface screen for specifying a particular study containing a slide desired to be reviewed by a user of a genotoxicity analysis system;

FIG. 12 illustrates an embodiment of a user interface screen for identifying the particular slide for review in the study selected in the user interface screen of FIG. 11;

FIG. 13 illustrates an embodiment of a user interface screen for displaying the results of screening of a particular slide using a particular genotoxicity test;

FIG. 14 illustrates an embodiment of a user interface screen that allows a user to retrieve objects that have been detected during an automatic scanning process using an automated genotoxicity testing system; and

FIGS. 15a through 15e present a listing of computer files for use in creating an embodiment of an automated genotoxicity testing system.

### DETAILED DESCRIPTION OF AN EMBODIMENT OF THE INVENTION

Described herein is a single automated platform for genotoxicity screening which can accommodate both in vivo and in vitro micronucleus testing, comet assay screening and in vitro metaphase finding, but requires minimal user monitoring and/or user interaction.

As will be more fully described, the present invention is an automated system and method for performing sample analysis for genotoxicity testing. An embodiment of the inventive system includes: (1) a robotic slide feeder, (2) an electronically driven microscope, (3) an image capturing apparatus, (4) a microprocessor-based computer running program control software, and (5) required communication cables and interface apparatus for interconnecting the various components. The invention is embodied in a system and method as exemplified in the embodiments described below, but is not limited to the details of those embodiments. One skilled in the art will readily appreciate that the invention may include and utilize equivalent components and processes that fall within the scope of the invention, which invention is defined solely by the claims that will accompany this disclosure. Moreover, the invention can comprise aspects of the foregoing components and their interrelationship to one another, including, without limitation, programmed control of such components.

Using the inventive automated genotoxicity analysis system and method, a laboratory technician or other user may optimally process, for analysis, successive batches of slides containing biological material using different tests and different types of biological material for each batch, without the need to manually adjust any hardware and with only minimal user interaction.

As will be more fully described below, the method of operation of the automated genotoxicity analysis system of the present invention proceeds as follows. A laboratory technician or other user prepares a batch of slides for genotoxicity screening. These slides may include in vivo or in vitro biological materials and may be prepared for screening by any of the following (or additional) tests: (1) in vivo micronucleus test, (2) in vitro micronucleus test, (3) in vitro or in vivo comet assay and (4) in vitro metaphase finding. Once the slides are prepared for testing, the user selects the appropriate genotoxicity test system (from the described list of possibilities) from a menu or equivalent user interface displayed on the screen of the microprocessor based computer. The robotic slide feeder then automatically retrieves the first of the slides from the batch prepared by the user and delivers the slide to the electronically driven microscope, which then automatically and appropriately focuses on the material contained on the slide. Next, the image capturing apparatus records a visual representation of the focused image and delivers it to the microprocessor-based computer. The microprocessor-based computer then performs image analysis on the recorded image using the appropriate image analysis software preloaded on the computer. The computer then records the data resulting from the analysis of the image until either the given delimiting number of cells have been counted or the maximum number of image fields to be analyzed has been reached for the slide currently under analysis. Once the analysis of the slide is complete, the robotic slide feeder returns the slide to the slide rack and retrieves the next slide for analysis. This process continues until all of the slides in the batch have been analyzed. The user may then prepare a new batch of slides of any type of in vivo or in vitro material and initiate automated screening of the material using any of the genotoxicity assays described above without the need to manually change or modify any of the system equipment.

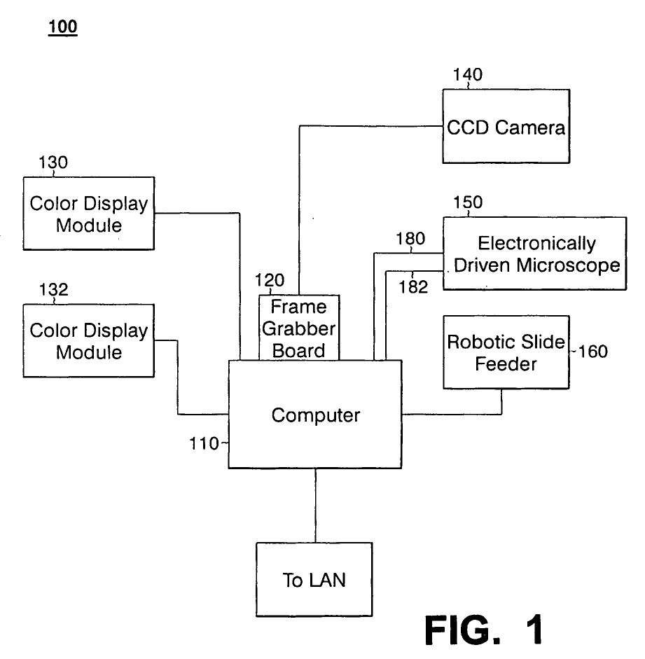

FIG. 1 illustrates, in block diagram form, an embodiment of the automated genotoxicity analysis system 100 of the present invention. Genotoxicity analysis system 100 includes a microprocessor-based computer 110 having a frame grabber board 120, two color display monitors 130 and 132, a charge coupled device (CCD) camera 140, an electronically driven microscope 150 and a robotic slide feeder 160.

Computer 110 of FIG. 1 may be any of the many known IBM-compatible personal or server computers running any known operating system for such computers, e.g., Windows XP, Windows NT Server or UNIX. In the preferred embodiment, a Transtec 1300 IBM compatible PC, operating at 1.3 GHz., having at least 128 Mbyte of internal RAM memory and running the Windows NT 4, Service Pack 5 operating system or Windows 2000 is utilized. Computer 110 executes all operating, control and image processing software, which will be described more fully below, for genotoxicity analysis system 100 and is connected to and controls the operation of all other components of genotoxicity analysis system 100. Computer 110 is connected to electronic miscroscope 150 and robotic slide feeder 160 via RS-232 serial interfaces. Computer 110 includes a frame grabber board 120 which is preferably a Meteor-II frame grabber utilizing Matrox MIL 6.1 or later version driver software available from Matrox Imaging of Dorval, Quebec. Computer 110 stores all program software and generated data on a local harddrive. Alternately, computer 110 may be connected to a local area network (LAN 200) to support data on a networked data base (not illustrated) or to allow access, retrieval and storage of parameter data files and other program software located on a separate networked computer server (not illustrated). In the preferred embodiment, the executable programs, compiled from the Visual Basic and C/C++ source code and the generated measurement data results files are stored on a networked database and server while the C-language DLLs and related files reside locally on the hard drive of computer 110.

Robotic slide feeder 160 is preferably an ES-553S robot with an SRC-320 driver available from Seiko Epson Corporation of Japan. Robotic slide feeder 160 is controlled and operated by electronic commands received from computer 110 via a serial cable 170. Robotic slide feeder 160 functions primarily to remove a current slide from a slide rack (not illustrated) containing a multiplicity of slides, then place the slide onto the stage of electronically driven microscope 150 and then return the slide to the slide rack after the analysis of the slide is complete. Under the embodiment of the invention described herein, the slide rack may include as many as 130 glass slides containing biological material or “samples.”

Electronically driven microscope 150 of genotoxicity analysis system 100 is preferably a Leica DM RXA/2 electronic microscope running Leica SDK driver software, which is manufactured and sold by Leica Microsystems AG of Wetzlar, Germany. Electronically driven microscope 150 preferably includes the following modules: stage, focus drive, illumination, objectives, fluorescence cubes, diaphragms for aperture and field, additional magnification changer and fluorescence shutter, all of which components are software driven and controllable. Electronically driven microscope 150 is controlled and operated by electronic commands received from computer 110 via two serial cables 180 and 182, one each for the stage controller and for the microscope stand of electronically driven microscope 150.

Camera 140 is preferably an XC-003 or DXC-390 CCD camera sold by Sony Corporation of America. Camera 140 is mounted on electronically driven microscope 150 in the known manner using a C-mount adapter and is utilized to grab the current image from electronically driven microscope 150 and send the image in analog format to frame grabber board 120 via serial cable 190. Camera 140 is under operational control of computer 110 via frame grabber 120. The analog formatted image received from camera 140 is digitized by computer 110.

Genotoxitiy analysis system 100 also includes color display modules 130 and 132 connected to computer 110. Preferably, color display module 130 provides the user interface to the user of the genotoxicity analysis system 100 while color display module 132 displays the current image provided by electronically driven microscope 150 or, alternatively, the result of the image processing analysis.

Computer 110 executes software which controls the operation of genotoxicity analysis system 100.

Computer 110, and any networked server that may also be utilized to control and operate genotoxicity analysis system 100, preferably runs Microsoft NT version 4 or Windows 2000 operating system software. The software executed by computer 110 to control genotoxicity analysis system 110 is created using Microsoft Visual Basic version 6 as well as Microsoft Visual C/C++ version 6. Annotated source code that may be utilized to create executable code as well as additional software and data files are attached as the Computer Program Listing Appendix for this documents and are described in greater detail below. One skilled in the art can implement the presently-described embodiment of the claimed genotoxicity analysis system, in part, by utilizing the software source code and related files in the Computer Program Listing Appendix and software available from third party providers.

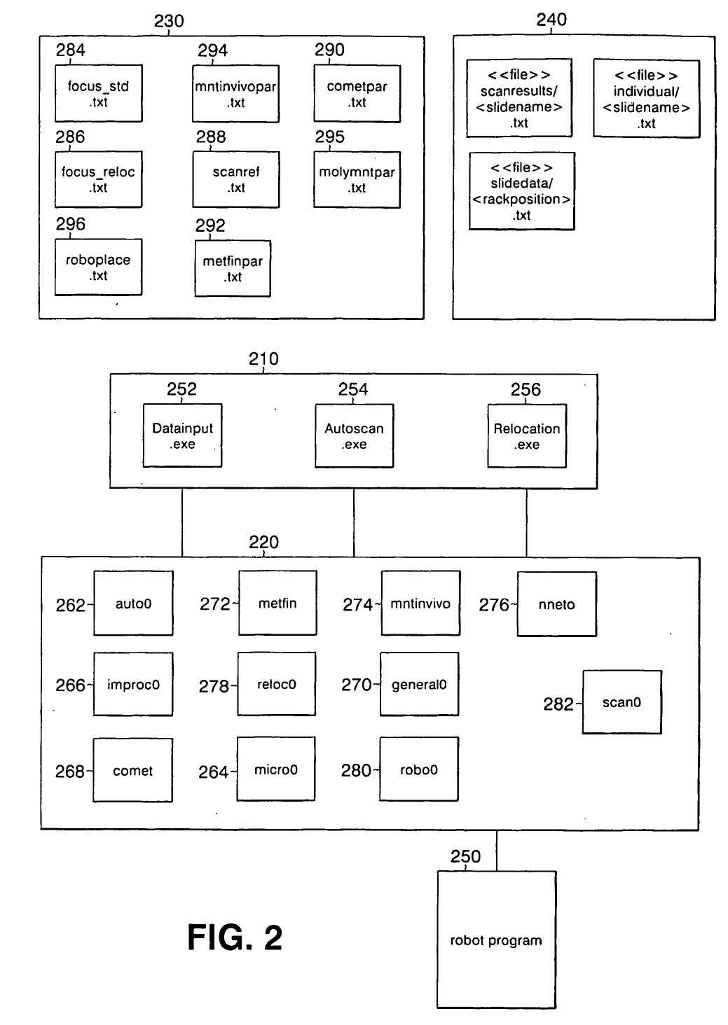

FIG. 2 illustrates, in logical block diagram form, a preferred embodiment of the application software 200 which resides in computer 110 and in a networked control server, to control the operation of genotoxicity analysis system 100 and also illustrates the files which store the results of the genotoxicity analysis. Application software 200 includes main executable programs 210, library link and DLL files 220, parameter files 230 and data results files 240, all of which will be described in greater detail below. Robot control program 250 is preferably control software provided by the manufacturer of robotic slide feeder 160.

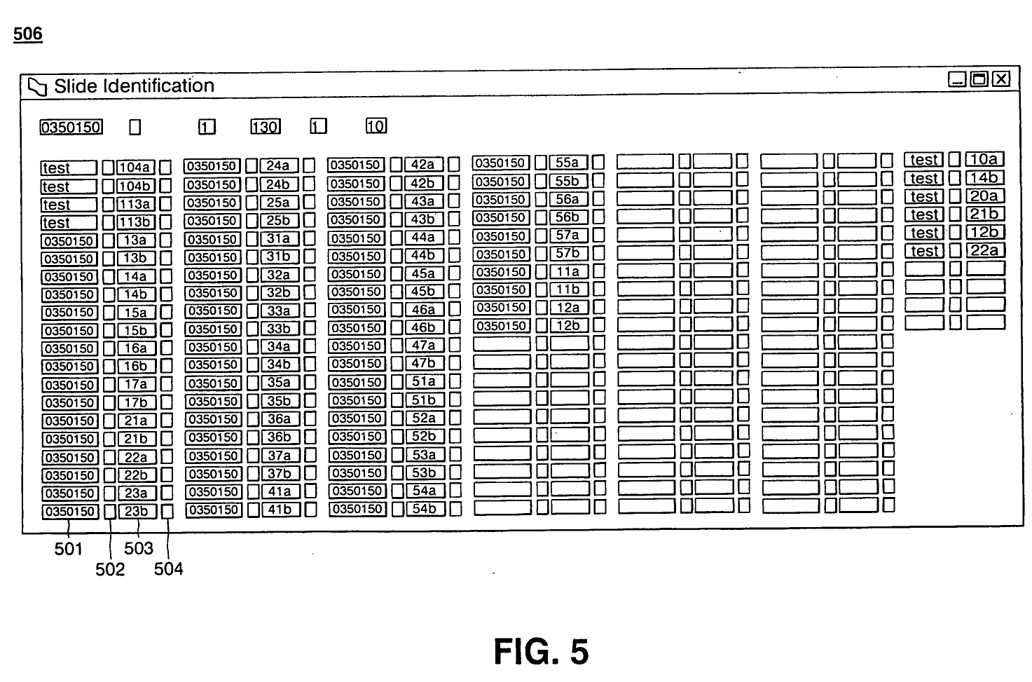

Main executable programs 210 include DataInput.exe 252, AutoScan.exe 254 and Relocation.exe 256. DataInput.exe 252 allows a user to enter information particular to each slide that is to be analyzed as shown, e.g., in FIG. 5 which will be explained below. AutoScan.exe 254 is used to initiate and provide fully automated selected genotoxicity screening of the slides that are identified using DataInput.exe 252. Relocation.exe 256 is a utility which allows a user to retrieve and manually view slides that have been processed using AutoScan.exe 254 in order to allow the user to visually inspect features of the biological material contained on the slide if necessary.

Executable programs 210 are each preferably compiled and linked to library link and DLL files 220 using Microsoft Visual Basic version 6.0. The source code for each of executable programs 210 references a respective file named “Globals.bas,” each version of which contains the respective “main” function for each of executable programs 210, and further includes other modules and necessary Visual Basic forms and code to create the various user interface windows. Also, as explained further below, executable programs 210 and the modules and forms associated with executable programs 210 operate by calling library link and DLL files 220 during operation.

The Computer Program Listing Appendix for this document includes the source code for creating each of executable programs 210 using Microsoft Visual Basic version 6.0. More particularly, the Computer Program Listing Appendix includes a folder named “VB6” which contains various subfolders. The subfolders named “DATAINPUT,” “AUTOMATICSCAN” and “RELOCATION” contain the source code for creating DataInput.exe 252, AutoScan.exe 254 and Relocation.exe 256, respectively.

The remaining subfolders in the folder labeled “VB6” in the Computer Listing Appendix contain source code for providing additional functionality for genotoxicity analysis system 100. These subfolders include “SUPERUSER” which stores source code for creating user interfaces that allow for manual adjustment of system parameters when necessary, “TOOLFORMS” which stores source code for user interface modules that may be used by executable programs 210, “PASSWORD,” which stores source code for providing password-protected access to genotoxicity analysis system 100 and “MODULES,” which includes source code for calling necessary library link and DLL files 220 during operation of genotoxicity analysis system 100.

In addition to the source code for creating executable programs 210, the Computer Program Listing Appendix for this document also includes source code for creating library link and DLL files 220 using Microsoft Visual C/C++ version 6.0. More particularly, the source code for generating library link and DLL files 220 is found in the subfolder labled “VC6” on the Computer Program Listing Appendix.

The subfolder labeled “AUTO0” in the folder named “VC6” contains source code for generating a C library called “auto0” 262 which provides functionality for facilitating the automatic functioning of genotoxicity analysis system 100, including autofocus control and automatic lamp adjustment, among others. The functionality provided by auto0 262 is based on the related functionality provided by the “micro0” 264 and “improc0” 266 DLLs which are described in greater detail below.

The subfolder labeled “COMET” in the folder named “VC6” contains source code for generating a C library called “comet” 268 which provides functionality required for performing image analysis on slides being analyzed for the comet assay.

The subfolder labeled “GENERAL0” in the folder named “VC6” contains source code for generating a C library called “general0” 270 which provides functionality for general purpose tools, including input and output functionality and graphic display routines.

The subfolder labeled “IMPROC0” contains source code for generating a C library called “improc0” 266 which provides interface functionality for the library of functions associated with the Matrox driver software of frame grabber board 120. These include functions relating to general image processing.

The subfolder labeled “METFIN” contains source code for generating a C library called “metfin” 272 which provides functionality required for performing image analysis on slides that are being analyzed for the metaphase finding application.

The subfolder labeled “MICRO0” contains source code for generating a C library called “micro0” 264 which provides interface and control functionality associated with the Leica SDK driver software for electronically driven microscope 150.

The subfolder labeled “MNTINVIVO” contains source code for generating a C library called “MNTinvivo” 274 which provides functionality required for performing image analysis on slides that are being analyzed for the micronucleus test in vivo application.

The subfolder labeled “NNET0” contains source code for generating a C library called “nnet0” 276 which provides functionality required for pattern classification through prediction using neural networks, e.g., the backpropagation algorithm, for the micronucleus test in vitro.

The subfolder labeled “RELOC0” contains source code for generating a C library called “reloc0” 278 which provides functionality for object retrieval within Relocation.exe 254, e.g., data input and output functionality and retrieval of analysis results.

The subfolder labeled “ROBO0” contains source code for generating a C library called “robo0” 280 which provides functionality required for communicating with robotic slide feeder 160.

The subfolder labeled “SCAN0” contains source code for generating a C library called “scan0” 282 which provides functionality required for facilitating an automatic scanning process, e.g., handling scanning mode settings, triggering the sequential analysis of the batch of slides to be processed and interfacing to specific application DLLs.

Additional libraries may also be included with library link and DLL files 220, including necessary library files provided by third party vendors for controlling operation of the electronically driven microscope 150 and frame grabber board 120.

The source code for certain of the above-described library link and DLL files 220 define the algorithm and image analysis processing that is conducted for the various screenings.

The image analysis processing for the micronucleus test in vivo uses red and blue camera channel information and thresholding techniques for discrimination between polychromatic and normochromatic erythrocytes. Thereafter, gradient and watershed transformation for segmentation of micronucleus candidates is utilized. Individual analysis of segmented objects uses supervised training of patterns on the basis of morphometric features, as well as structural features such as “periphery percentage,” “focus deviation” and “gray deviation.” Reference may be made to the applicable source code described above for further detail.

Metaphase finding utilizes differences of spectral images as the gray image basis and thereafter utilizes a combination of watershed transformation and “top-hat” segmentation for nucleus candidate segmentation. That is followed by restriction of metaphase range on non-nuclear regions which is followed thereafter with another application of top-hat and watershed segmentation. Finally, feature base metaphase candidate classification, involving individual parameters for chromosomal structuring, is applied. Reference may be made to the applicable source code described above for further detail.

Comet assay analysis involves red channel uses of fluorescence image on a first run to detect valid nuclei, including classification on morphometric features. Automatic relocation of detected nuclei for tail moment measurement and use of a sequentially degrading thresholding technique which involves a gradient for the pixel sum change in the image is also utilized. Reference may be made to the applicable source code described above for further detail.

The micronucleus test in vitro uses all three color channel images. The image algorithms attempt segmentation of valid nuclei and cytoplasm range, and then detect micronucleus candidates using a combination of gradient, top-hat and thresholding segmentation. Final classification uses an off-line trained backpropagational neural network for predicting the probability of a true micronucleus. Reference may be made to the applicable source code described above for further detail.

Continuing with FIG. 2, application software 200 further includes parameter files 230 which store information about the proper settings for the operation of electronically driven microscope 150 and the image analysis software operating on genotoxicity analysis system 100, depending upon the particular analysis being conducted. Each of the parameters and adjustments varies depending on the genotoxicity test to be conducted and is set automatically by designating the particular test.

Parameter files 230 include the following files:
“cometpar.txt” 290—contains parameters for the configuration of the image analysis algorithms used for the Comet assay application;
“metfinpar.txt” 292—contains parameters for the configuration of the image analysis algorithms used for the metaphase finding application;
“mntinvivopar.txt” 294, contains parameters for the configuration of the image analysis algorithms used for the micronucleus test in vivo application; and
“molymntpar.txt” 295 contains parameters for the configuration of the image analysis algorithms used for the micronucleus test in vitro application.

Parameter files 230 further include a file called “focus_std.txt” 284 which contains parameter data that controls the automatic focus features of electronically driven microscope 150 in connection with the autofocus execution for Datainput.exe 252 and AutoScan.exe 256. Parameter file 230 called “focus_reloc.txt” 286 generally contains the same parameter definitions as “focus_std.txt” 284, but is more refined to allow for autofocus performance that is better suited for operation under Relocation.exe 254. Parameter file 230 labeled “scanref.txt” 288 contains parameter data that is used for the configuration of electronically operated microscope 150 depending on the selected application. Such configuration includes automatic adjustment of optical modules of the microscope, and setting general parameters referring to the scanning process of the application.

Also, the parameter file 230 called “roboplace.txt” 296 contains parameter data to control the initialization and placement of robotic slide feeder 160. These parameters include x,y positioning and speed.

Each of “focus_std.txt” and “focus_reloc.txt,” are particularized for the screening test being performed, i.e., there exists a “focus_std.txt” and “focus_reloc.txt” for each of the in vivo micronucleus test, in vitro micronucleus test, comet assay or in vitro metaphase finding. Computer Program Listing Appendix stores the parameter files 230 for each screening type in respective file folders.

More particularly, Computer Program Listing Appendix includes a folder named “Applications” which includes subfolders labeled “COMETASSAY” containing the above described parameter files 230 used for comet assay analysis. Similarly, the subfolder called “METFIN” contains the above described parameter files for metaphase finding analysis. The subfolder called “MNTINVIVO” contains the above described parameter files for in vivo micronucleus test analysis.

The subfolder called “MOLYMNT” contains the above described parameter files for in vitro micronucleus test analysis. The “MOLMNT” subfolder further includes a file called “p21h9.net” and includes parameters for the neural network pattern prediction and classification utilized for the in vitro micronucleus test analysis.

In a preferred embodiment, “robias.txt,” which holds system specific information for the application in general for genotoxicity analysis system 100 and “roboplace.txt” 296, which contains parameters for use by robotic slide feeder 160 during initialization, reside locally on the hard drive of computer 110 while the remaining programs and files reside on a networked server connected to computer 110.

In addition to the above-described parameter data files, calibration files containing “shadimages”, including “shadref_black” and “shadref_whitbl”, are referenced by the executable programs 210. One skilled in the art may generate these files to provide calibration for shading correction. Calibration files are particular to each screening application. The calibration files are preferably stored in a subdirectory that is parallel to the respective subdirectories containing the parameter data.

Application software 200 of FIG. 2 further includes data results files 240 which are generated and modified by executable programs 210.

There are three types of data results files 240 having the following forms:
(1) <<path>>scanresults/<study>/<experiment>/<slidename>.txt;
(2) <<path>>individualdata/<study>/<experiment>/<slidename>.txt; and
(3) <<path>>slidedata/slidedata<rackposition>.txt
   In the above-listed file formats for data results files 240, <<path>> indicates the preliminary file path of the directory containing the file at issue. This part of the path may vary depending upon how the file structure of the overall operational software is configured. “scanresults,” individualdata” and “slidedata” represent respective subfolder names for the files. <study> represents a placeholder for the study name coding the toxicological testing of a certain test compound and is correlated with a unique “study name”, <experiment> represents a placeholder for a particular experiment in the context of the selected study. Experiments belonging to a specific study can vary with respect to treatment time or the absence or presence of the metabolic activation of cells, or sampling time after treatment of animals. Generally, it specifies the “experimental” conditions for the same test compound of interest. <slidename> represents a placeholder for the identity of a particular slide and <rackposition> represents a placeholder for a particular position of a slide in a rack.

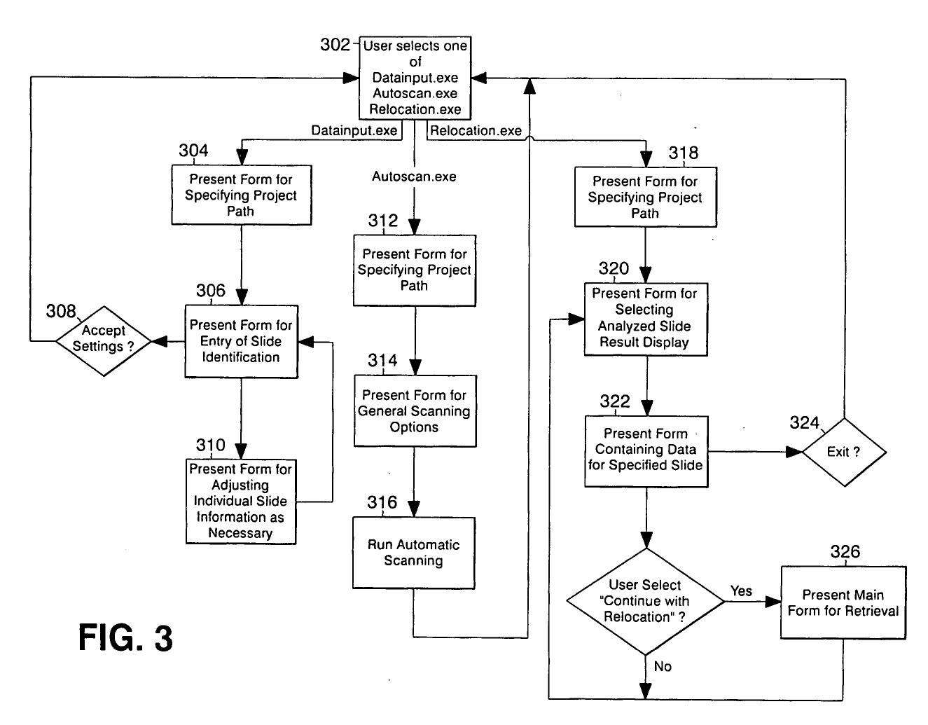

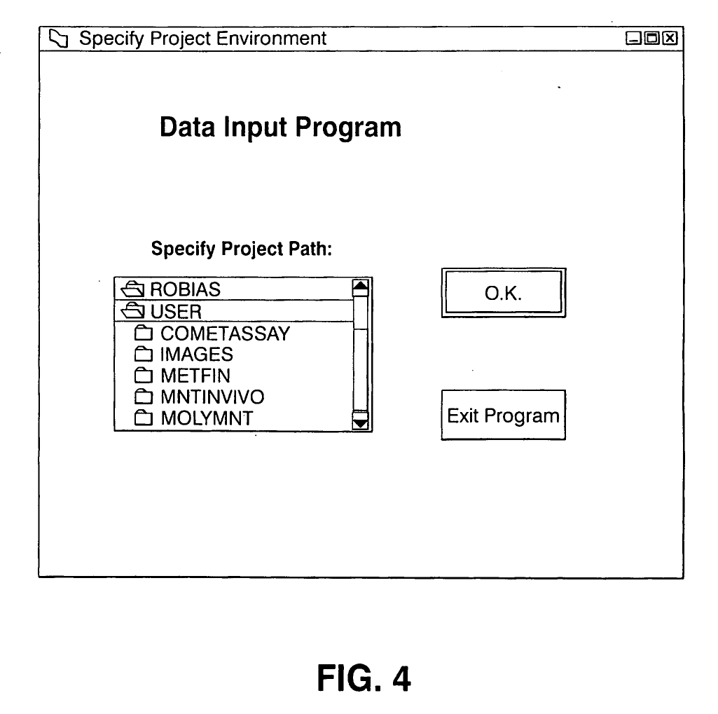

The operation of genotoxicity analysis system 100 will now be described with reference to the flow chart of FIG. 3 and the exemplary screen interfaces of genotoxicity analysis system 100 illustrated in FIGS. 4 through 14.

At step 302 of the process of FIG. 3, the user selects one of DataInput.exe, AutoScan.exe and Relocation.exe for execution from the main display screen of color display monitor 130. Each application is preferably represented as an application or shortcut icon on the main display screen of the Windows NT platform. The user may select the desired program by double clicking the corresponding icon in the known manner.

If the user desires to enter information for each slide that is to be analyzed, the user selects the icon representing DataInput.exe for execution at step 302. As a result, the process proceeds to step 304 where the form illustrated in FIG. 4 is displayed to the user. Using the form of FIG. 4, the user specifies the current application, i.e., the analysis that is to be performed, by selecting a unique path to which the slide data will be written. Thus, selection of the path also designates the analysis that will be performed, i.e., comet assay, micronucleus test in vivo, micronucleus test in vitro or metaphase finding. The form of FIG. 4 is created from the source code found in the file called “frmInit.frm” in the VB6/TOOLFORMS subdirectory of the Computer Program Listing Appendix for this document.

The process then moves to step 306 where the form illustrated in FIG. 5 is displayed to the user. Using this form, the user enters data for identifying each slide that is to be processed. The identification string for each slide consists of a study name (col. 501), followed by experiment name (col. 502) and a slide code (col. 503), each of which may utilize numerals or characters. It is noted that the exemplary slide codes 503 presented in FIG. 5 are appended by “a” and “b.” In the presently disclosed embodiment of the present invention, two samples of biological material may be included on each slide, one designated by “a”, the other by “b.” The precision provided by the components of genotoxicity analysis system 100 in combination with application software 200 allows for this efficient use of slide space which effectively doubles slide capacity for screening.

For slides sharing the same study and experiment code, a common folder for resulting storage will be created. The form of FIG. 5 is created from source code found in the file called “frmSlides.frm” in the VB6/DATAINPUT subdirectory of the Computer Program Listing Appendix for this document.

At step 308, the user accepts the settings entered at step 306 by pressing the “Accept settings” button 506 of the form of FIG. 5, at which point the system ends operation of DataInput.exe, creates all necessary folders (for studies and experiments) and data files and returns to step 302 of FIG. 3.

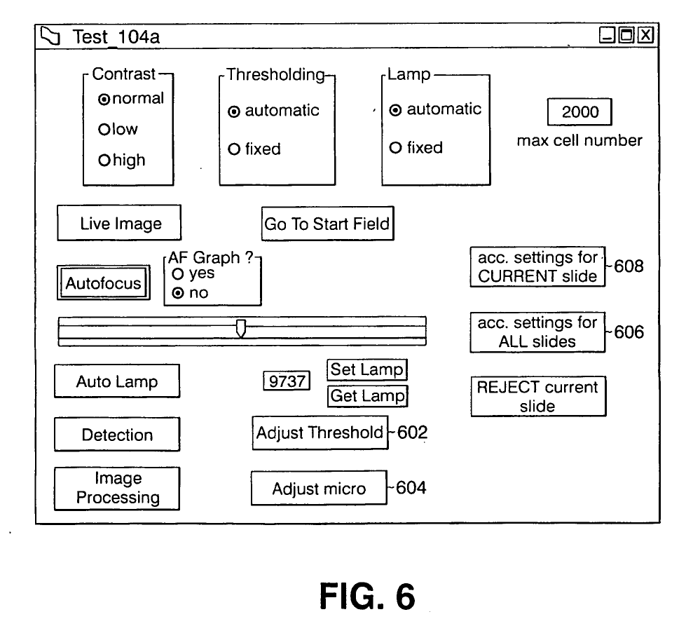

Alternately, at step 310, the user may select any of the respective detail buttons (see column 504 of the form of FIG. 5) for each slide to adjust specific parameters relating to each slide. FIG. 6 represents the form presented to the user for adjusting the parameters for a particular slide. The form of FIG. 6 is created from the source code found in the file called frmSlideparam.frm in the VB6/DATAINPUT subdirectory of the Computer Program Listing Appendix for this document.

Among the various parameters that the form of FIG. 6 allows a user to control is threshold adjustment (button 602) and microscope adjustment (button 604).

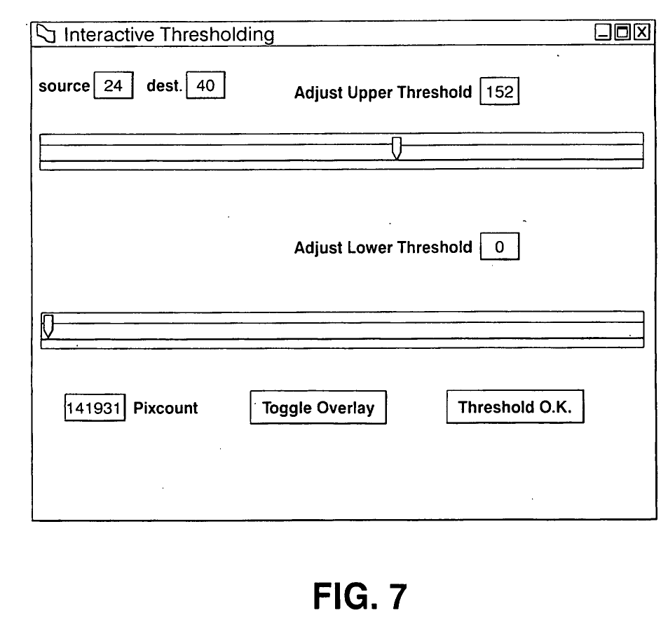

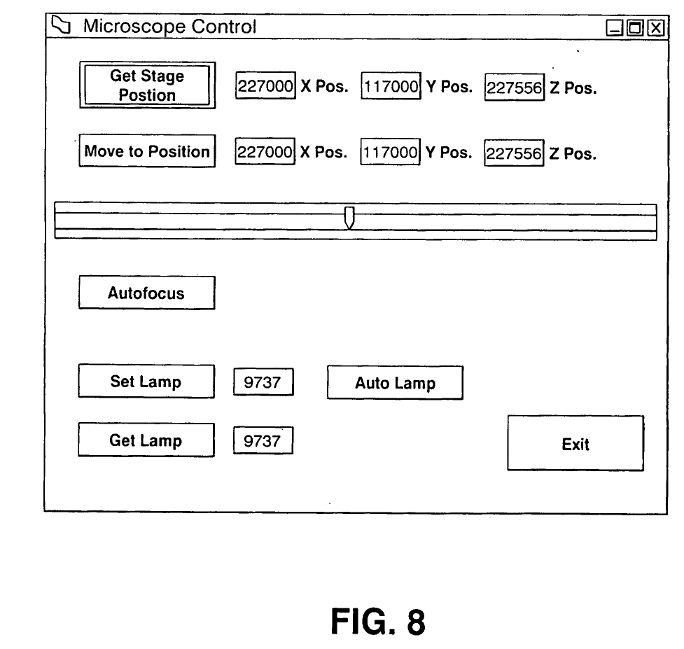

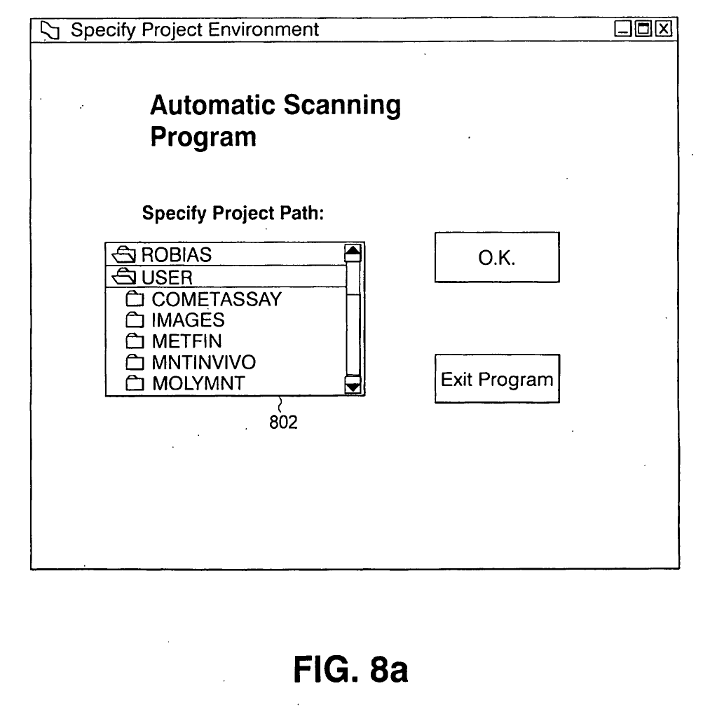

The form for providing the user the ability to adjust the threshold settings for the particular slide is illustrated in FIG. 7. This form is created from the source code found in the file called “frmInterThresh.frm” in the VB6/TOOLFORMS subdirectory of the Computer Program Listing Appendix for this document. The form for providing the user the ability to adjust microscope parameters for the particular slide at issue is illustrated in FIG. 8. This form is created from source code found in the file called “frmAdjustMicro.frm” in the VB6/TOOLFORMS subdirectory of the Computer Program Listing Appendix for this document.

Once the user is satisfied with the adjustments made to the particular slides, the user may select the “acc. Settings for ALL slides” button 606 of the form of FIG. 6 which will set these parameters for all previously identified slides that have valid slide code entries in the form of FIG. 5. Alternatively, the user may select the “acc. settings for CURRENT slide” (button 608) which saves parameter settings only for the currently selected slide. Control is then returned to the form of FIG. 5 (step 306).

Returning now to step 302 of the process illustrated in FIG. 3, if the user selects the icon to initiate execution of AutoScan.exe, the process moves to step 312 where the form illustrated in FIG. 8a is presented to the user. Here, the user selects the genotoxicity test that will be processed, i.e., one of the comet assay, micronucleus in vivo, micronucleus in vitro or metaphase finding analysis, by selecting the respective subdirectory illustrated in window 802 in the form of FIG. 8a. The form of FIG. 8a is created from source code found in the file called “frmInit.frm” in the VB6/TOOLFORMS subdirectory of the Computer Program Listing Appendix for this document.

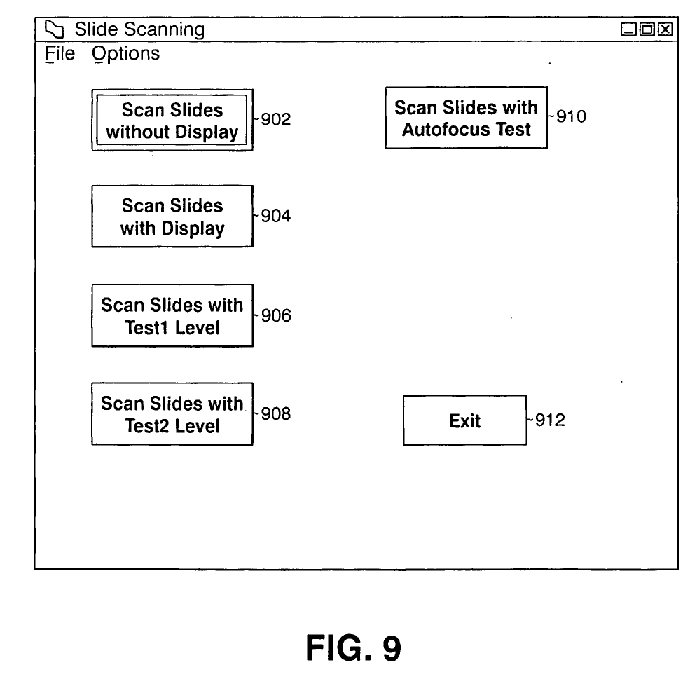

The process then proceeds to step 314 where the form of FIG. 9 is presented to the user. The form of FIG. 9 allows the user to select the scanning options for the genotoxicity analysis to be performed as was specified at step 312 using the form of FIG. 8. The options presented by the form of FIG. 9 include: (1) scanning the slides without display (button 902), meaning that no intermediate image display will be presented to the user during analysis of the slides; (2) scanning the slides with display (button 904), meaning that the most important intermediate image processing results will be displayed during analysis without requiring user interaction to continue analysis; (3) scanning the slides with Test1 level (button 906), meaning that several intermediate image processing steps are performed and the process is then halted until the user presses a key to continue automatic analysis; and (4) scanning the slides with Test2 level (button 908), which results in operation similar to that of button 906 except that detection results are not displayed. This last mode is utilized to validate operation of the application where a user performs manual analysis of a slide in parallel with automated analysis in the same image fields. Finally, the user may press button 910 for scanning the slides with autofocus test, which processes the slides while presenting a graphical display of the autofocus results, e.g., contrast curve, for each slide.

The user may also abort running the analysis by pressing the exit button 912.

If the user does not abort the automatic scanning, the process proceeds to step 316 and the automatic scanning is executed by referencing the applicable library link and DLL files 220 and parameter files 230 of application software 200 for the specific type of analysis being performed. The form of FIG. 9 is created from source code found in the file called “frmMain.frm” in the VB6/AUTOMATICSCAN subdirectory of the Computer Program Listing Appendix for this document.

When the automatic scanning is complete and all results data has been written and stored, the process returns to step 302 of FIG. 3.

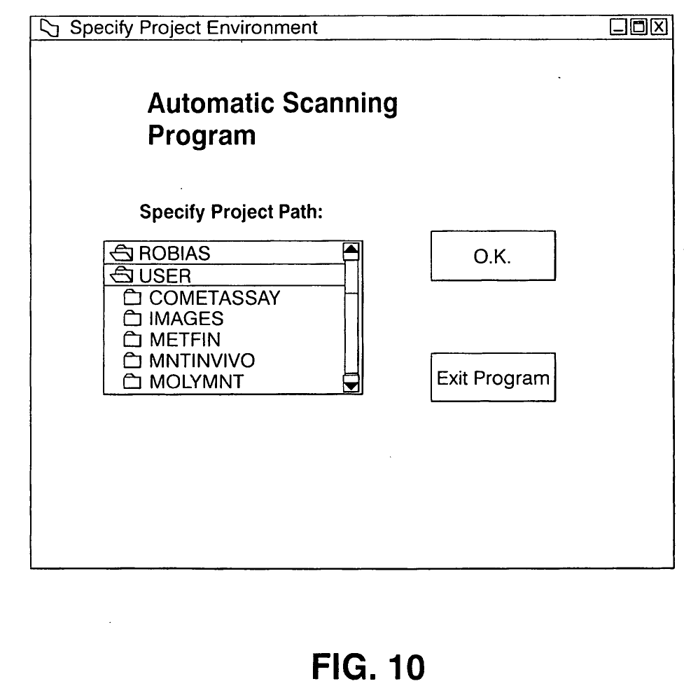

If at step 302, the user executes Relocations.exe, the process of FIG. 3 proceeds to step 318 where the form of FIG. 10 is presented to the user. Using the form of FIG. 10, the user selects the genotoxicity test for which results are to be reviewed, i.e., one of the comet assay, micronucleus in vivo, micronucleus in vitro or metaphase finding analysis, by selecting the respective subdirectory illustrated in window 1002 in the form of FIG. 10. The form of FIG. 10 is created from the source code found in the file called “frmInit.frm” in the VB6/TOOLFORMS subdirectory of the Computer Program Listing Appendix for this document.

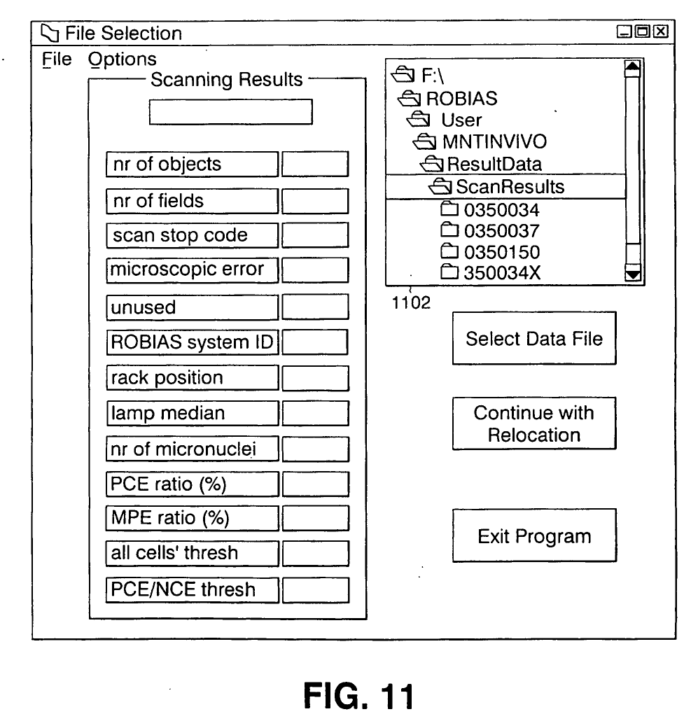

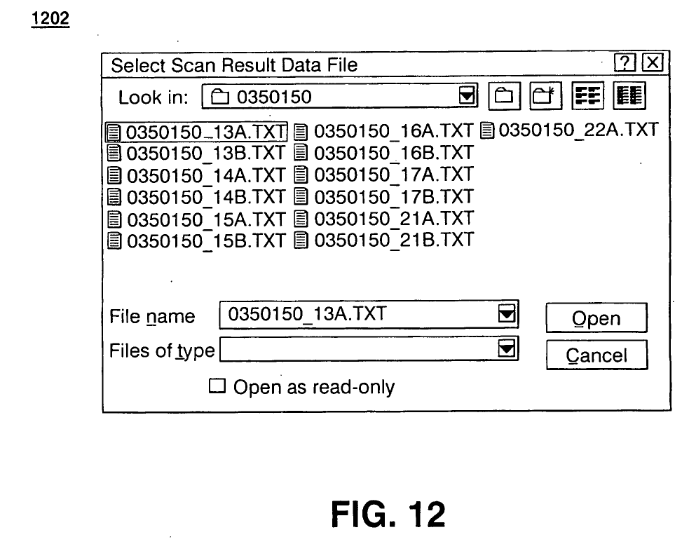

The process then proceeds to step 320 where the user is presented with forms to select a specific slide to be reviewed. More particularly, the user is presented with the forms illustrated in FIGS. 11 and 12. In the form of FIG. 11, the user selects the particular study containing the slide by selecting the appropriate subdirectory labeled with the appropriate study and experiment name (see window 1102). Using the form of FIG. 12, the user identifies the particular slide by selecting the file containing the slide data (see window 1202). The form of FIG. 11 is created from source code found in the file called “frmMain.frm” in the VB6/RELOCATION subdirectory of the Computer Program Listing Appendix for this document. The form of FIG. 12 is created using a standard Visual Basic CommonDialog user interface object.

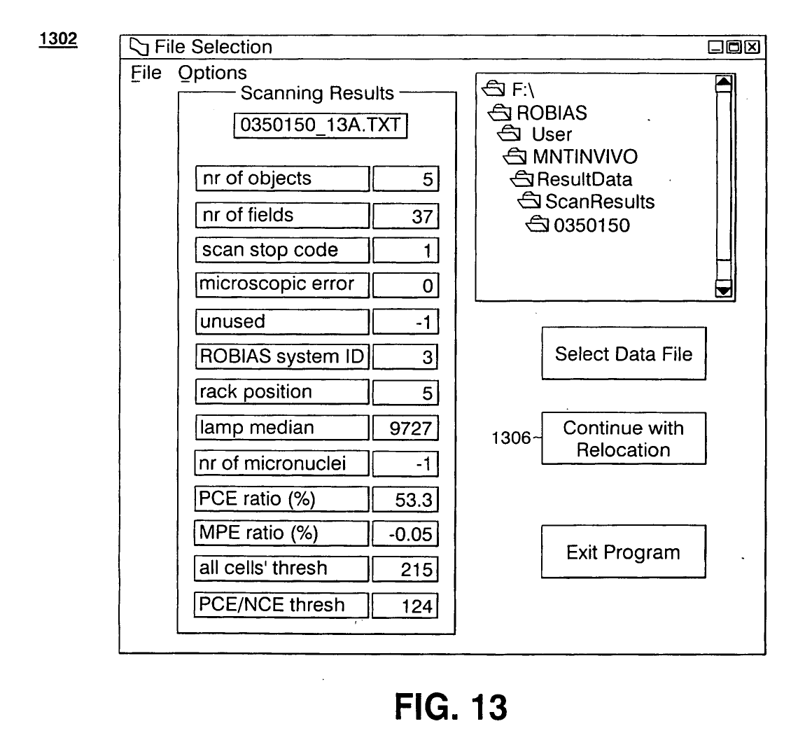

Next, at step 322, the user is presented with the form of FIG. 13 which includes a display (window 1302) of the scanning results associated with the slide specified using the form of FIG. 12. The form of FIG. 13 is similar to that of FIG. 11 except that it now includes, in window 1302, the most relevant data that had been acquired during automatic slide analysis, such as the number of detected objects, number of scanned fields, error codes, and other application specific information for the slide under review.

The user may exit Relocation.exe by clicking button 1304 of the form of FIG. 13 (step 324) at which point the process of FIG. 3 returns to step 302.

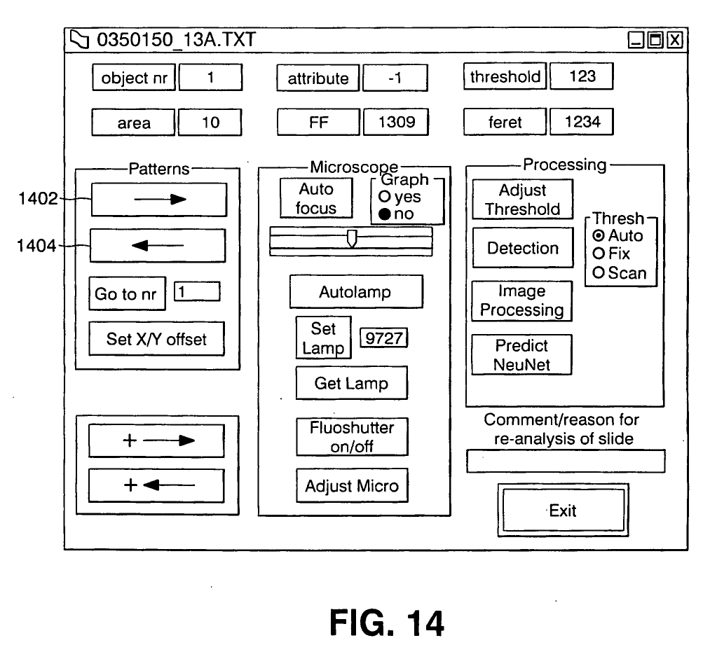

Alternatively, the user may select button 1306 of the form of FIG. 13 causing the process of FIG. 3 to move to step 326 at which point the user is presented with the form of FIG. 14. The form of FIG. 14 allows a user to retrieve the objects that had been detected during the automatic scanning process. For this purpose, one can move from one object to another (and then back again) using the arrow buttons 1402 and 1404. Using the additional controls presented in the form of FIG. 14, each object's coordinates, which had been stored during scanning, and the current live image showing the object are displayed on color display screens 130 and 132 for visual inspection. The user may operate the right or left mouse button to flag an object under observation and discard an object as a valid micronucleus (by using the left mouse button) or accept an object as a valid micronucleus (using the right mouse button). By moving from the first detected object to the last for each slide, the user can assign the proper label (i.e., “accept” or “reject”) to each object and, therefore, adjust the result of automatic scanning through supervised visual inspection. The corrected result for the current slide, i.e. the number of micronuclei for micronucleus application, or number of metaphases for metaphase finding application, will be stored after exiting the form of FIG. 14. The other options present in the form in FIG. 14 support the adjustment of the current image, e.g., microscope and focus, and support image analysis for other objects of interest in order to confirm proper performance of the algorithms utilized for image analysis.

The form of FIG. 14 is created from source code found in the file called “frmRelocation.frm” in the VB6/RELOCATION subdirectory of the Computer Program Listing Appendix for this document.

Thus, it is seen by the above, that by creating software code which can facilitate different types of genotoxicity screening and which references parameter data files respectively configured for each of various genotoxicity tests, the genotoxicity analysis system of the present invention provides a flexible and easy to use platform for performing various genotoxicity screenings with minimal user interaction. Depending upon the type of screenings being performed, no manual microscope module adaptation is necessary between screening runs for different analysis testing. In the case of comet assay screening, a manual change to incident illumination to support fluorescence staining in comet assay analysis and then back to transmitted light illumination for other genotoxicity screenings may be necessary. Moreover, as described above, the genotoxicity analysis system of the present invention allows interactive pattern control to permit a user to manually perform artifact rejection for objects wrongly classified during automatic scanning.

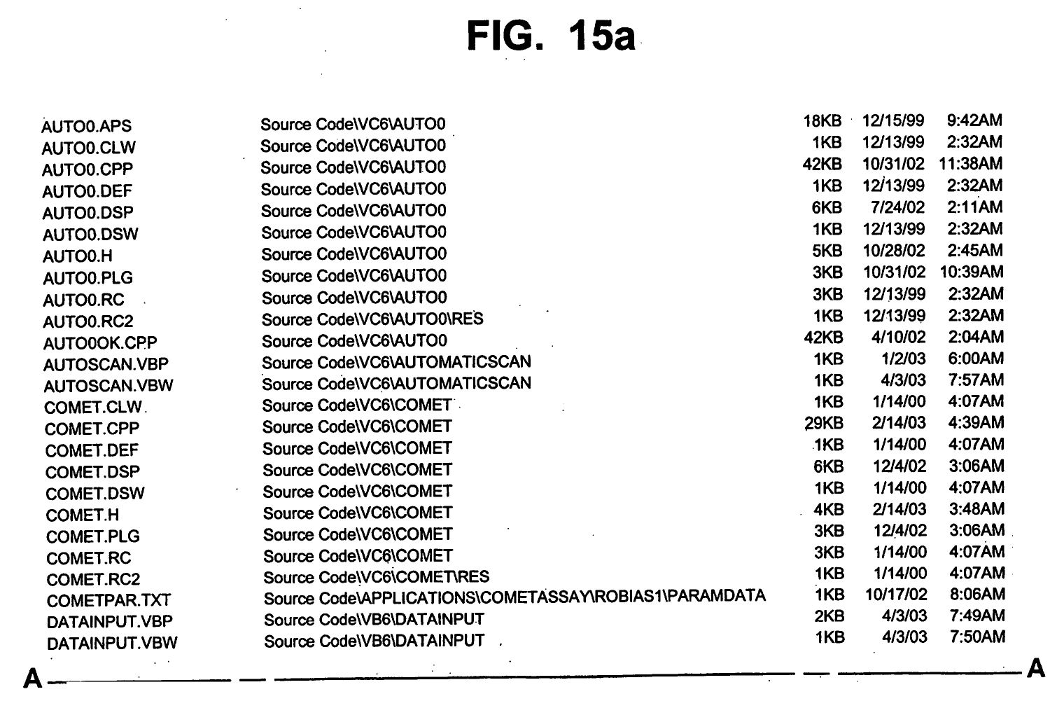

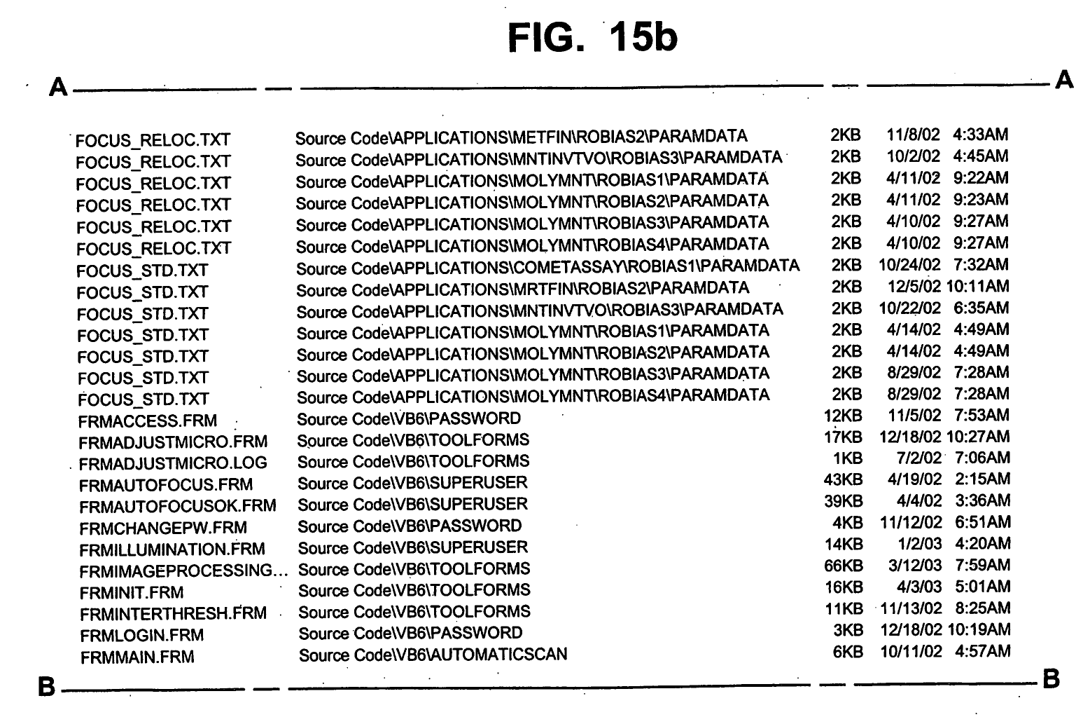

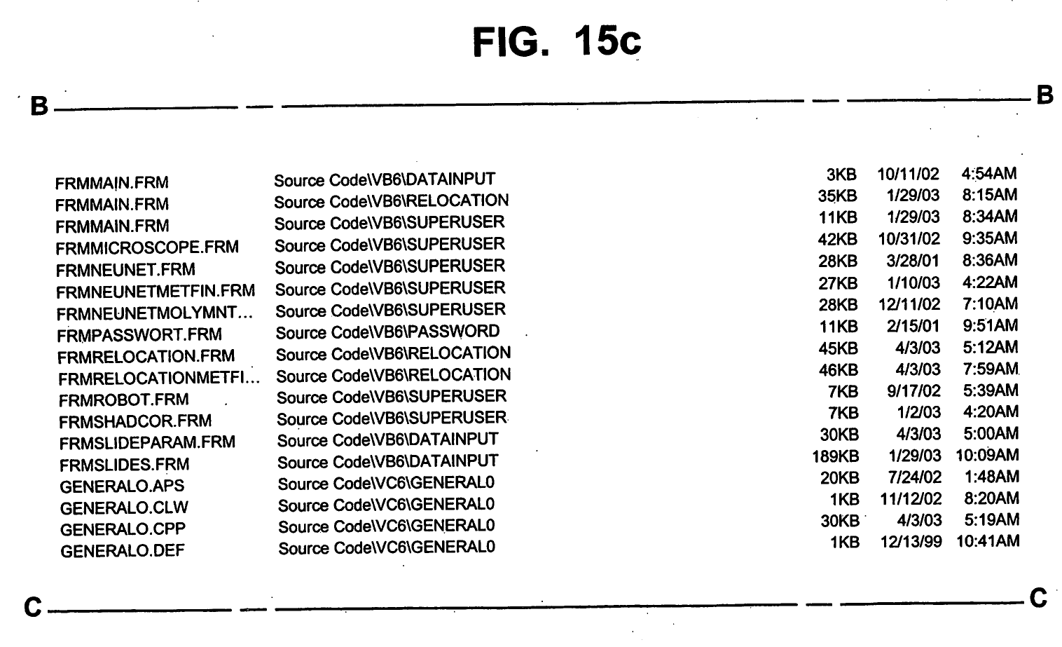

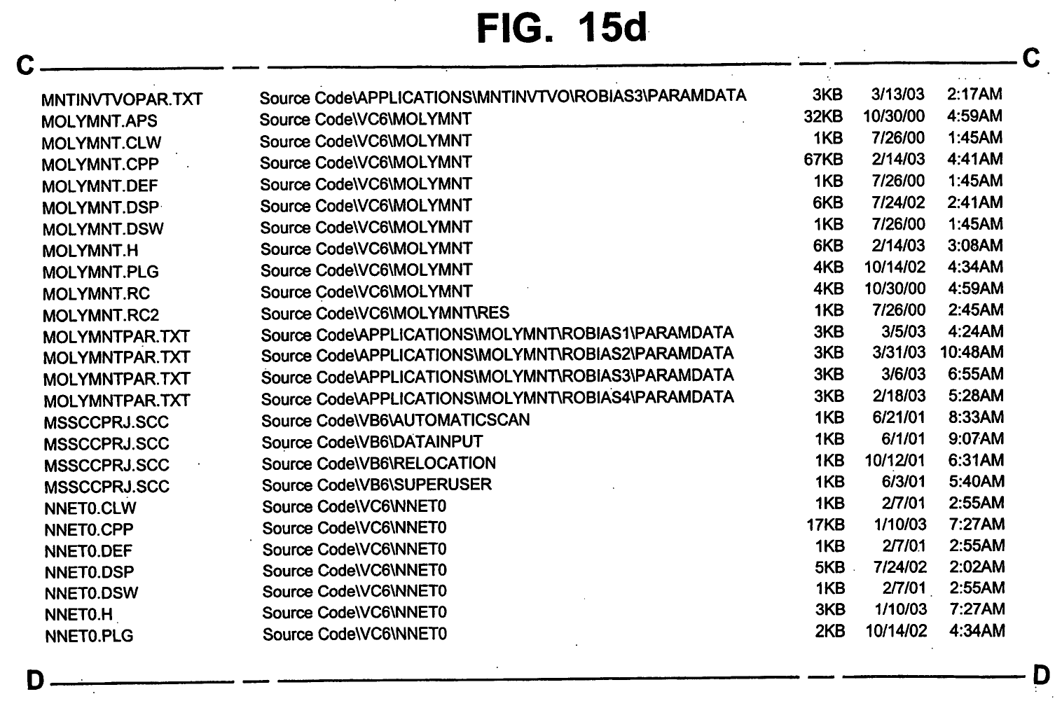

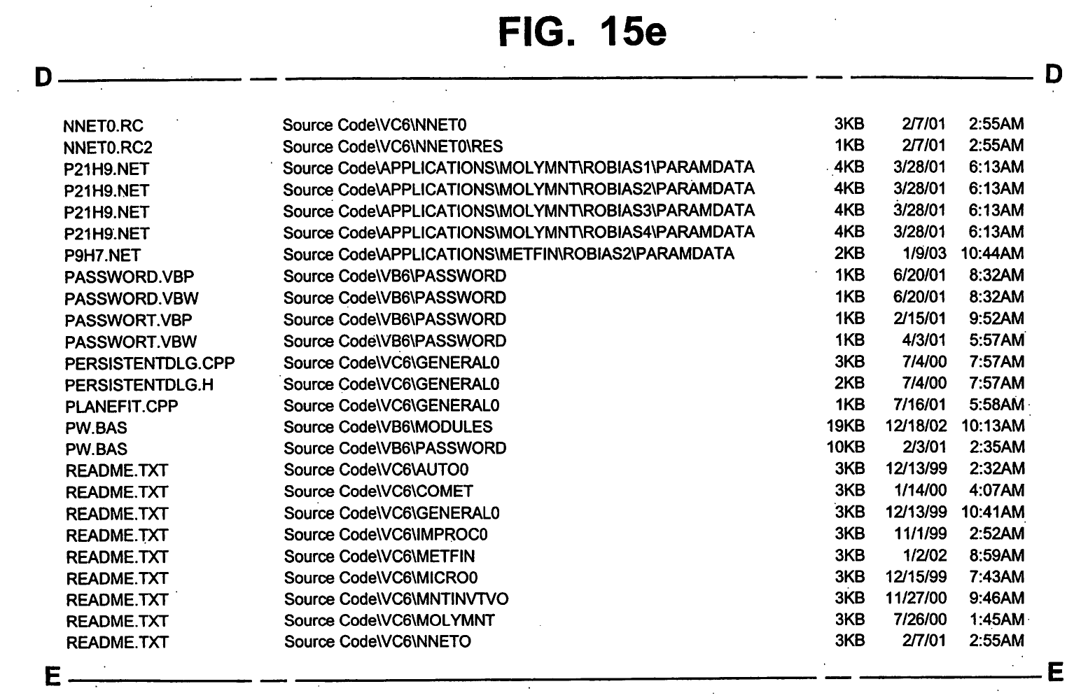

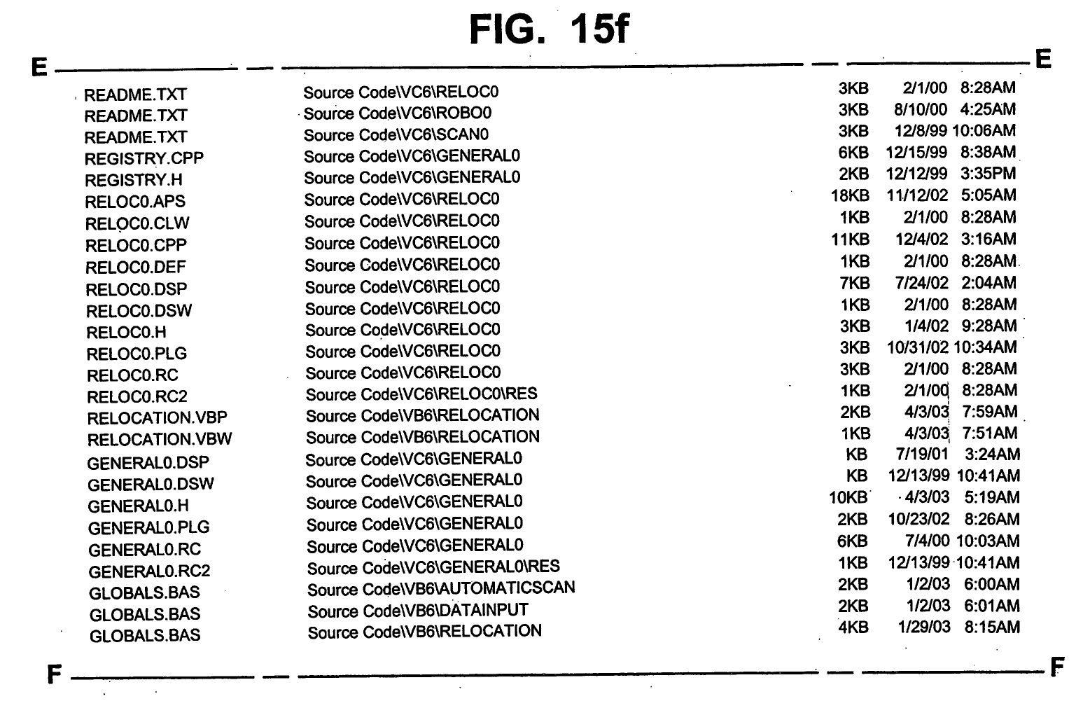

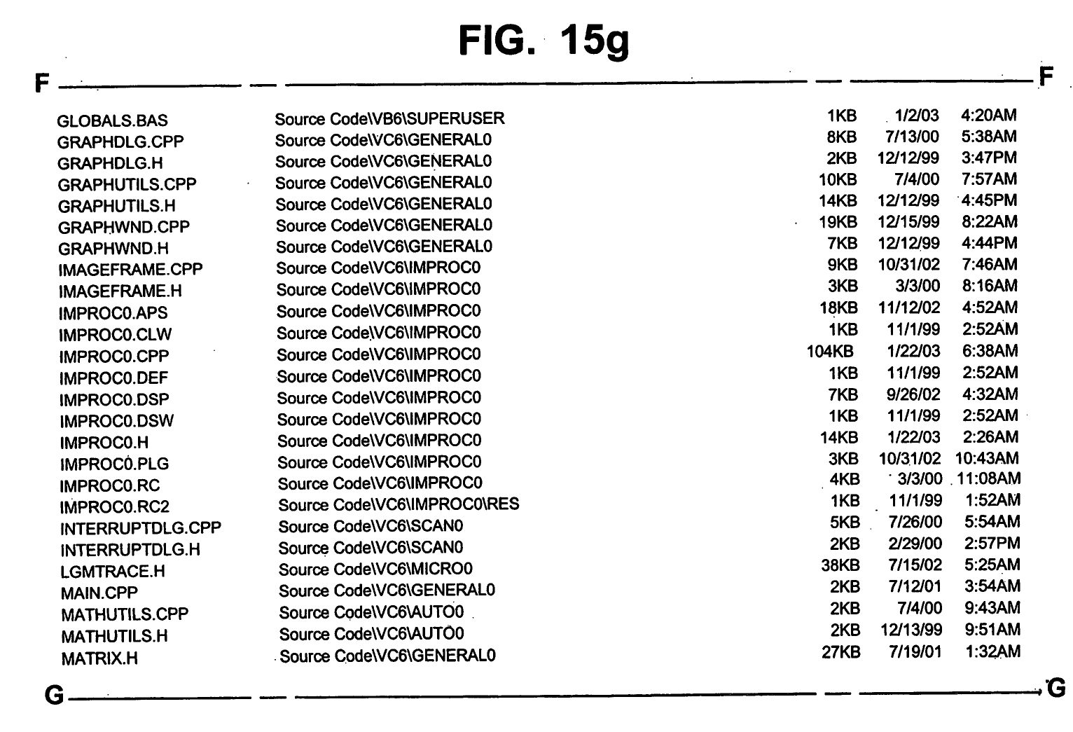

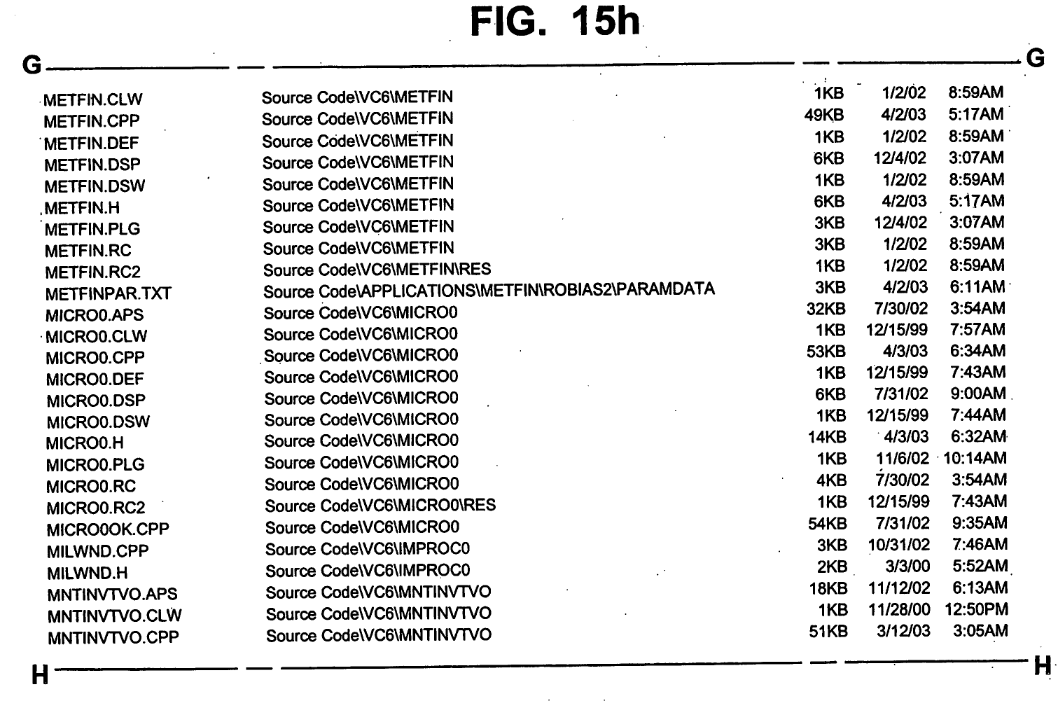

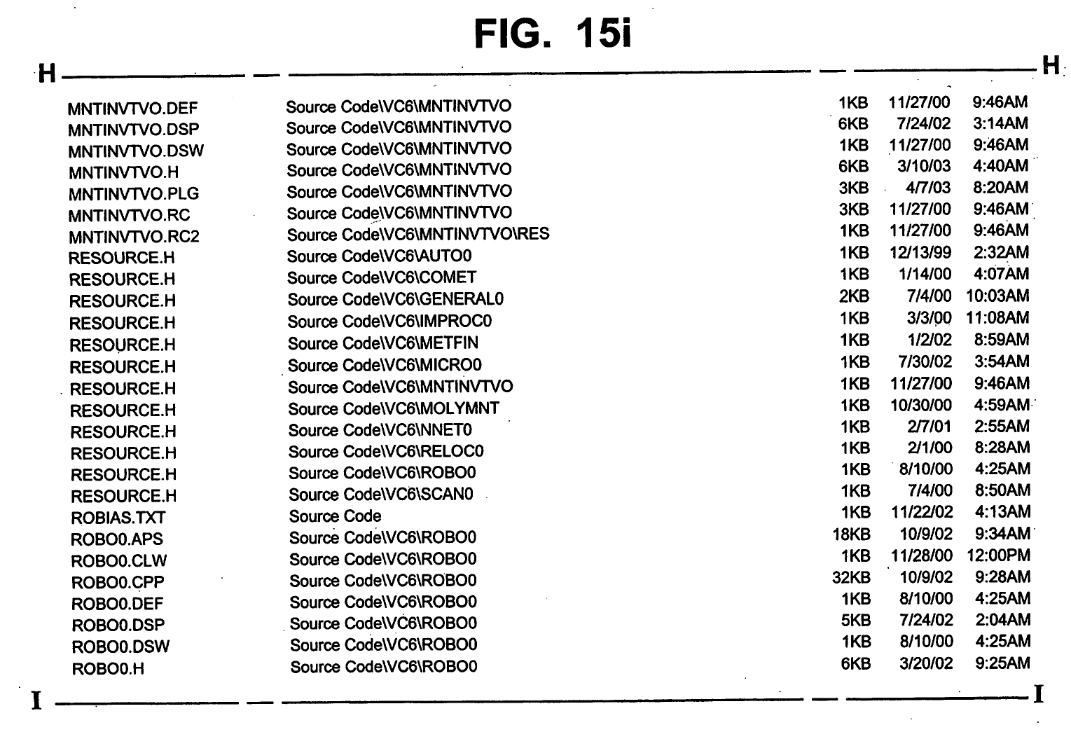

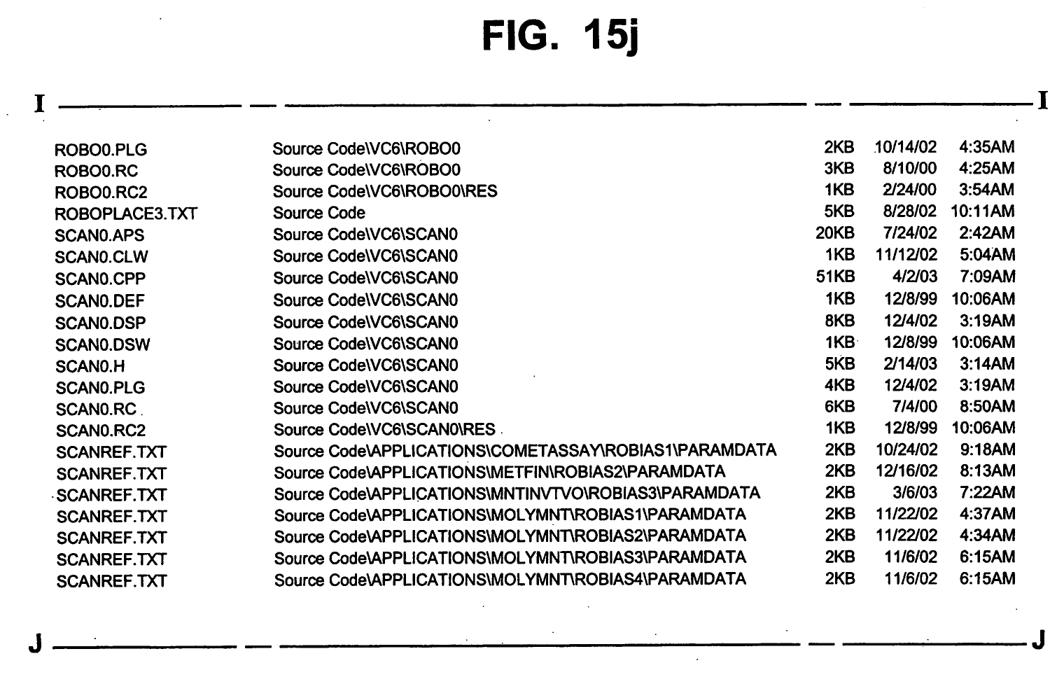

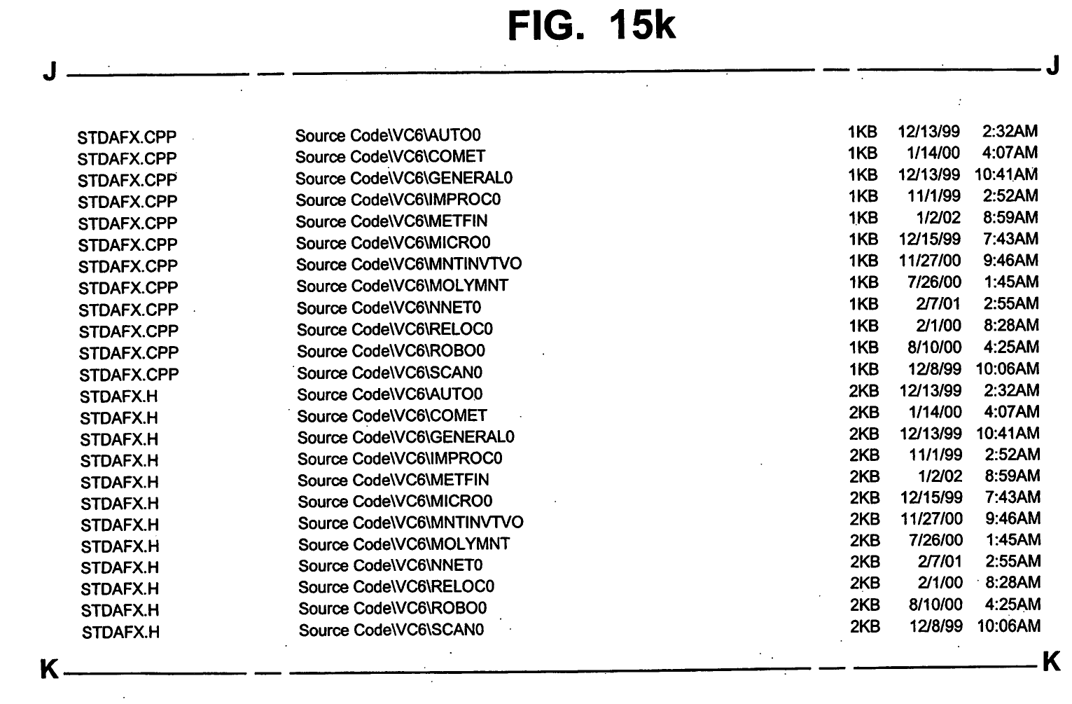

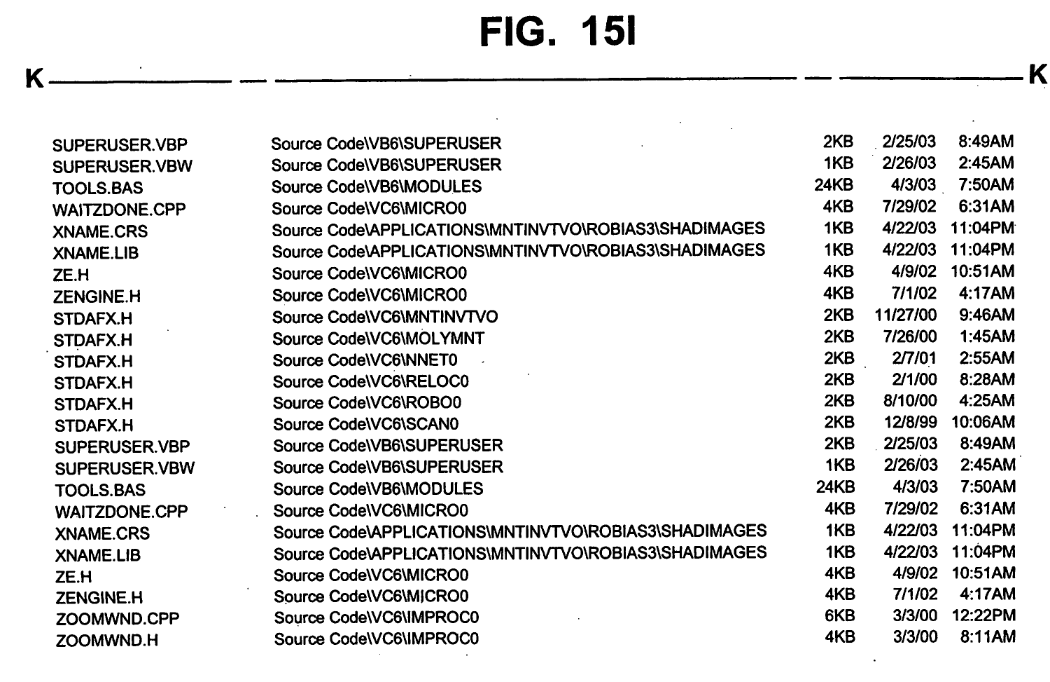

In accordance with 37 C.F.R. 1.52 (e), the name, respective creation date and size (in bytes), of each file contained on the CD-ROM of the Computer Program Listing Appendix are listed in FIGS. 15a-15. For ease of reference, the file names are listed as they appear in the directory structure of the Computer Program Listing Appendix.

I claim:

1. A system for providing genotoxicity screening, the system comprising:
a. one or more computers;
b. a frame grabber connected to the one or more computers;
c. a camera connected to the frame grabber;
d. a microscope connected to the one or more computers; and
e. a slide feeder connected to the one or more computers;
f. a program operating on the one or more computers operative to facilitate the screening a second batch of biological material using a second genotoxicity testing method after screening a first batch of biological material using a first genotoxicity testing method, substantially free of any manual manipulation of the camera, the microscope or the slide feeder.

2. The system of claim 1, further including a user interface presented on a display monitor connected to the one or more computers, for allowing a user of the genotoxicity screening system to select the genotoxicity screening method to be performed on a given batch of biological material.

3. The system of claim 1, the camera, the microscope and the slide feeder including physical connections that receive electronic signals from the one or more computers which control the operation of the camera, the microscope and the slide feeder.

4. Software for controlling the operation of a genotoxicity analysis system, the software providing automatic configuration of configurable components of the genotoxicity analysis system and allowing the genotoxicity analysis system to perform a plurality of genotoxicity tests on respective pluralities of biological samples by way of the automatic configuration.

5. The software of claim 4, wherein the software allows a user to specify the genotoxicity test to be performed on a given group of biological samples.

6. The software of claim 5, wherein, after the user has specified the genotoxicity test to be performed, the software automatically generates signals which are sent to the configurable components of the genotoxicity analysis system in accordance with the specified genotoxicity test.

7. The software of claim 6, wherein the sent signals cause the configurable components of genotoxicity analysis system to be configured in a manner conducive to the selected genotoxicity test.

8. The software of claim 4, the software further providing a user of the genotoxicity analysis system with the ability to provide identifying information for each biological sample.

9. The software of claim 4, the software functioning to record the results of the genotoxicity testing for each analyzed sample and providing the further functionality of allowing manual inspection of the recorded results of the genotoxicity testing.

10. The software of claim 4, the software including respective files containing data defining configurable parameters of the configurable components for each of the plurality of genotoxicity test.

11. The software of claim 4, the software containing software code defining respective image analysis techniques for use by each of the plurality of genotoxicity tests.

12. A method for performing genotoxicity testing of biological materials by utilizing a genotoxicity analysis system including hardware components that are operated with software controls, the genotoxicity analysis system being capable of performing a multiplicity of genotoxicity tests, the method comprising the steps of:
preparing a first batch of samples of biological materials for processing using a first genotoxicity test;
utilizing the genotoxicity analysis system to perform a first genotoxicity test on the samples of the first batch of biological materials;
preparing a second batch of samples of biological materials for processing using a second genotoxicity test;
utilizing the genotoxicity analysis system to perform a second genotoxicity test on the samples of the second batch of biological materials,
wherein the software controls manipulate the configuration of the hardware components during the time period between performance of the first and second genotoxicity tests to thereby allow the first and second genotoxicity tests to be performed using the same hardware components.

13. A method for performing various types of genotoxicity tests on respective batches of biological samples using a genotoxicity analysis system, the method including the steps of:
a. receiving a command from a user of the genotoxicity analysis system, the command specifying the type of genotoxicity test to be performed;
b. performing an automatic configuration of the component of the genotoxicity analysis system to thereby allow the genotoxicity analysis system to perform the genotoxicity test specified in step a;
c. performing the specified genotoxicity test on a batch of biological samples;
d. recording results of the genotoxicity test;
e. repeating steps a through d.

14. The method of claim 13, wherein the types of genotoxicity tests are selected from the group consisting of one or more of the following: the micronucleus test in vivo, the micronucleus test in vitro, the comet assay and metaphase finding.

15. A method for performing genotoxicity screening comprising the steps of:
a. preparing a batch of slides for genotoxicity screening;
b. selecting a genotoxicity test;
c. automatically retrieving the first of a plurality of slides containing biological samples from a slide retaining device;
d. automatically delivering the slide to an electronically driven microscope;
e. automatically focusing on the material contained on the slide;
f. automatically recording a visual representation of the focused image;
g. automatically delivering the focused image to a microprocessor-based computer;
h. automatically performing image analysis on the recorded image using image analysis software appropriate for the genotoxicity test selected in step b.
i. automatically recording the data resulting from the analysis of the image;
j. automatically returning the slide retrieved in step c to the slide retaining device;
k. automatically retrieving the next slide for analysis;
l. automatically repeating steps c through k for successive slides in the batch until all of the slides in the batch have been analyzed; and
m. repeating steps a through l until all desired slides have been processed.

16. The method of claim 15, including the further step of manually verifying the recorded data.

17. The method of claim 15, wherein the batch of slides is prepared in accordance with the genotoxicity test to be performed, the genotoxicity test being selected from the group consisting of: the micronucleus test in vivo, the micronucleus test in vitro, the comet assay and metaphase finding.

18. The method of claim 15, wherein the selecting step is performed by choosing the appropriate test from a menu displayed on a video monitor.

19. The method of claim 15, wherein the steps of automatically retrieving and automatically returning is performed by a robotic slide feeder.

20. The method of claim 15, wherein the step of automatically recording the data resulting from the analysis of the image is continuously performed until either a given delimiting number of cells have been counted or the maximum number of image fields to be analyzed has been reached for the slide currently under analysis.
